(19) United States
(12) Patent Application Publication
(10) Pub. No.: US 2004/0099840 A1
(43) Pub. Date: May 27, 2004

(54) Oxygen scavenging compositions

(76) Inventors: Mark A. Horsham, Mascot, New South Wales (AU); James K G Murphy, Baulkham Hill, New South Wales (AU); Andrew D Scully, North Ryde, New South Wales (AU)

Correspondence Address: MCDERMOTT WILL & EMERY, 600 13TH STREET, N.W., WASHINGTON, DC, 20005-3096, US

(21) Appl. No.: 10/451,248

(22) PCT Filed: Dec. 21, 2001
(86) PCT No.: PCT/AU01/01652

(30) Foreign Application Priority Data
Dec. 22, 2000 (AU) PR 2296

(51) Int. Cl. C09K 3/00; C07D 303/12

(52) U.S. Cl. 252/188.28; 549/556

(57) ABSTRACT

Epoxy anthraquinonesulfonamide compounds are disclosed having the Formula (I) wherein X¹, X², X³, X⁴, X⁵, X⁶, X⁷ and X⁸ are each independently selected from H, OH, NH₂, C₁-C₄₀ alkyl, C₁-C₄₀ alkoxy, C₁C₄₀ alkanoyl, C₁-C₄₀ alkanol, C₁-C₄₀ alkylether, C₁-C₄₀ alkylthio, C₁-C₄₀ alkylamino, C₁-C₄₀ alkanolether, C₁-C₄₀ alkylaminoether, C₁-C₄₀ alkylsulfonyl, C₁-C₄₀ alkyl sulfonanmido, epoxy sulfonamido substituents, and sulfonate substituents, with the proviso that at least one of X¹, X², X³, X⁴, X⁵, X⁶, X⁷ and X⁸ is an epoxy sulfonamido substituent, and salts thereof. The compounds and reaction products thereof may be used in oxygen scavenging compositions.

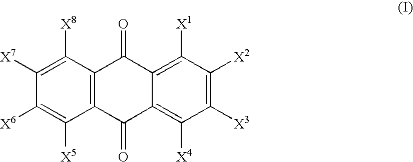

### FIELD OF THE INVENTIONS

This invention relates to oxygen scavenging compositions comprising a new class of anthraquinone compounds for use in, for example, food and beverage packaging to scavenge unwanted oxygen, which either remains within the package following the packaging of the food or beverage or otherwise enters the package by permeating through the packaging material. The new class of anthraquinone compounds may also be incorporated into packaging materials to prevent oxygen from permeating through the packaging material to enter the inside of a package. Furthermore, the new class of anthraquinone compounds may also be incorporated into packaging materials to reveal leaks in packages or to indicate package damage caused by handling or tampering.

### BACKGROUND TO THE INVENTION

A wide variety of foods, beverages and other materials are susceptible to loss in quality if they are exposed to significant amounts of oxygen during storage. The damage can arise from, for example, chemical oxidation of the product and/or microbial growth. In the field of packaging, such damage has been traditionally addressed by generating relatively low-oxygen atmospheres by vacuum packing and/or inert gas flushing. However, these methods are not generally applicable for various reasons. For example, the fast filling speeds commonly used in the food and beverage industries often prevent effective evacuation of, or thorough inert gas flushing of, food and beverage packages, and neither evacuation or inert gas flushing provides any residual capacity for removal of oxygen which may have desorbed from the package contents or entered the package by leakage or permeation. As a consequence, there has been much interest in the identification and development of chemical techniques for generating low-oxygen atmospheres.

In Australian Patent No. 672661 (the entire disclosure of which is incorporated herein by reference), the present applicants describe novel oxygen scavenging compositions comprising anthraquinone (AQ) compounds or oligomers or polymers including AQ moieties, which may be readily activated or “triggered” (i.e. brought to their oxygen scavenging form) as required by exposure to, for example, ultraviolet (TV) light. The oxygen scavenging compositions, once activated, are capable of scavenging oxygen from an oxygenated atmosphere or liquid in substantial darkness for periods ranging from up to a few minutes or hours to over 100 days. Numerous specific applications for the oxygen scavenging compositions are disclosed in that patent as well as in the applicant's co-pending Australian Patent Application No. 87230/98 (the entire disclosure of which is incorporated herein by reference).

Epoxy sulfonamido compounds have been reported previously for use, for example, as hardeners and in the preparation of epoxy resins (see, for example, European Patent No. 0265753 and European Patent No. 0339137). It has been realised by the present applicants that such epoxy sulfonamido compounds may be linked to an AQ moiety to provide reaction products which may be utilised, either alone, or in a composition with other compounds and/or substances, to provide materials suitable for use in/as oxygen scavenging packaging.

### DISCLOSURE OF THE INVENTION

Thus, in a first aspect, the present invention provides a compound of the following formula:

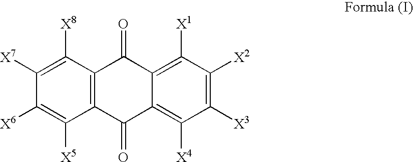

wherein X¹, X², X³, X⁴, X⁵, X⁶, X⁷ and X⁸ are each independently selected from H, OH, NH2, C₁-C₄₀ alkyl, C₁-C₄₀ alkoxy, C₁-C₄₀ alkanoyl, C₁-C₄₀ alkanol, C₁-C₄₀ alkylether, C₁-C₄₀ alkylthio, C₁-C₄₀ alkylamino, C₁-C₄₀ alkanolether, C₁-C₄₀ alkylaminoether, C₁-C₄₀ alkylsulfonyl, C₁-C₄₀ alkyl sulfonamido, epoxy sulfonamido substituents, and sulfonate substituents, with the proviso that at least one of X¹, X², X³, X⁴, X⁵, X⁶, X⁷ and X⁸ is an epoxy sulfonamido substituent,

and salts thereof.

Preferably, X¹, X², X³, X⁴, X⁵, X⁶, X⁷ and X⁸ are each independently selected from H, OH, NH₂, C₁-C₂₀ alkyl, C₁-C₂₀ alkoxy, C₁-C₂₀ alkanoyl, C₁-C₂₀ alkanol, C₁-C₂₀ alkylether, C₁-C₂₀ alkylthio, C₁-C₂₀ alkylamino, C₁-C₂₀ alkanolether, C₁-C₂₀ alkylaminoether, C₁-C₂₀ alkylsulfonyl, C₁-C₂₀ alkyl sulfonamido, epoxy sulfonamido substituents, and sulfonate substituents, with the proviso that at least one of X¹, X², X³, X⁴, X⁵, X⁶, X⁷ and X⁸ is an epoxy sulfonamido substituent,

The epoxy sulfonamido substituent(s) is/are preferably selected from those having the following formula:

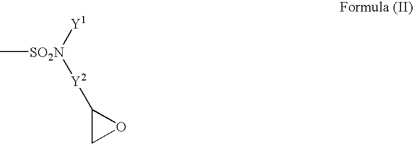

wherein Y¹ is selected from H, C₁-C₄, alkyl, C₁-C₄C alkanol, C₁-C₄₀ alkanolether, C₁-C₄₀ alkylamine, C₁-C₄₀ alkylaminoether, C₁-C₄₀ alkylether, C₁-C₄₀ aryl, C₁-C₄₀ arylalkyl and

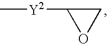

and Y² is selected from C₁-C₂₀ alkyl.

Preferably, Y¹ is selected from H, C₁-C₂₀ alkyl, C₁-C₂₀ alkanol, C₁-C₂₀ alkanolether, C₁-C₂₀ alkylamine, C₁-C₂₀ alkylaminoether, C₁-C₂₀ alkylether, C₁-C₂₋₀ aryl, C₁-C₂₀ arylalkyl and

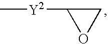

and preferably, Y² is selected from C₁-C⁶ alkylene. More preferably, Y² is methylene.

Where the compound includes two epoxy sulfonamido substituents, said substituents may be the same or different. Where the compound includes three or more epoxy sulfonamido substituents, two or more of said substituents may be the same or, alternatively, all may be different.

Preferably, only one of X¹, X², X³ and X⁴, and/or one of X⁵, X⁶, X⁷ and X⁸ is/are an epoxy sulfonamido substituent(s).

Most preferably, the compound is selected from the group consisting of 2-(N,N-diglycidyl) anthraquinonesulfonamide, 2-(N-butyl-N-glycidyl)-anthraquinonesulfonamide and 2,6-(N,N′-dibutyl-N,N′-diglycidyl)-anthraquinonesulfonamide.

The compound of the first aspect may be reacted with any compound or compounds containing one or more nucleophilic groups, such as amines or acids, to produce reaction products which may be utilised, either alone, or in a composition with other compounds and/or substances, to provide materials suitable for use in/as oxygen scavenging packaging.

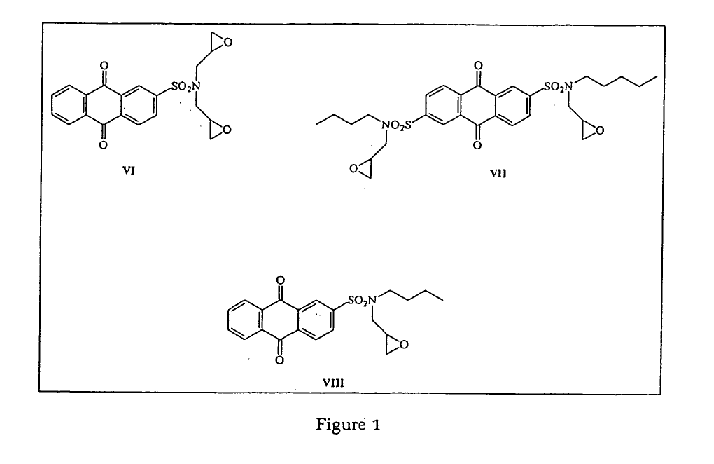

Thus, in a second aspect, the present invention provides a reaction product of a compound according to the first aspect (such as compounds of formula VI and VII shown in FIG. 1), and a dinucleophilic compound.

Preferably, the dinucleophilic compound is selected from primary amines, bis(secondary) diamines, dihydric phenols, dicarboxylic acids, anhydrides, diols, dithiols and disulfonamides.

More preferably, the reaction product of the second aspect is a compound of the following formula:

B¹-[-(Z¹-Aⱼ^{l})ₖ-(Z²-Aⱼ²)ₗ- . . . -(Zⁿ-Aⱼⁿ)ₘ-]-B² Formula (III)

wherein Z¹, Z², . . . , and Zⁿ are each independently selected from the group consisting of radicals derived from di-epoxy compounds (such as the diglycidyl ether of bisphenol A), and where at least Z¹ is selected from the group consisting of radicals derived from an epoxy anthraquinonesulfonamide (preferably, radicals derived from compounds of formula VI and VII in FIG. 1), according to the first aspect,

A¹, A², . . . and Aⁿ are each independently selected from the group consisting of radicals derived from dinucleophilic compounds selected from primary amines, bis(secondary) diamines, dihydric phenols, dicarboxylic acids, anhydrides, diols, dithiols and disulfonamides.

B¹ is selected from the group consisting of an epoxy group, or remnants of an epoxy group after reaction with Aⁿ, or suitable capping groups (i.e. Monofunctional reactants capable of reaction with epoxy groups such as carboxylic acids, secondary amines and monohydric phenols. Examples of monofunctional reactants are acetic acid, benzoic acid, diethanolamine, N-(2hydroxyethyl)-piperazine and phenol.). B² is selected from the group consisting of H, or an epoxy group, or radicals derived from di-epoxy compounds (such as the diglycidyl ether of bisphenol A) or their remnants after reaction with Aⁿ, or suitable capping groups.

k is 1, l is 0 or 2, m is 0 or any integer in the range of 3 and 1000, and n is equal to m, wherein when m is other than 0, 1 must be 2, and

j is 0 or 1 when l and m are 0, and j is 1 when l and m are other than 0.

The values of the subscripts k, l and m are not intended to indicate any particular ordering of the associated units.

Thus, where m is 3, the reaction product is:

B¹-[-(Z¹-A¹)-(Z²-A²)-(Z³-A³)-(Z⁴-A⁴)-]-B² Formula (IV)

and where m is 4, the reaction product is:

B¹-[-(Z¹-A¹)-(Z²-A²)-(Z³-A³)-(Z⁴-A⁴)-]-B² Formula (V)

etc.

All or some of Z¹, Z², . . . and Zⁿ may be the same or different, and/or all or some of A¹, A², . . . and Aⁿ may be the same or different.

The reaction product of the second aspect may provide a polymer capable of scavenging oxygen when used on its own, or when used in combination with other compounds and/or substances to provide an oxygen scavenging composition.

In a third aspect, the present invention provides a reaction product of a compound according to the first aspect (such as compound VIII as shown in FIG. 1), and a compound containing one or more nucleophilic groups.

Preferably, the nucleophilic compound contains one or more amine, acid, anhydride, phenol, alcohol, thiol or sulfonamide groups.

The reaction product of the third aspect may result from a reaction with a nucleophilic compound selected to provide improved compatibility enabling the reaction product to be blended with other compounds and/or substances to provide an oxygen scavenging composition.

In a fourth aspect, the present invention provides an oxygen scavenging composition comprising a compound according to the first aspect or a reaction product according to the second or third aspect.

Preferably, the composition further comprises a hydrogen donor compound such as, for example, a compound containing a hydrogen bonded to a nitrogen, sulfur, phosphorus or oxygen, especially where a hydrogen is bonded to a carbon atom bonded to one of the abovementioned heteroatoms. Alternative sources of hydrogen are salts of organic compounds such as the salts of sulfonic acids or carboxylic acids.

In addition, the composition preferably comprises an activated oxygen scavenging agent, i.e. an agent which reacts with activated oxygen species such as peroxide. Suitable activated oxygen scavenging agents include organic antioxidants, organic phosphites, organic phosphines, organic phosphates, hydroquinone and substituted hydroquinone; inorganic compounds including sulphates, sulphites, phosphites and nitrites of metals; sulphur-containing compounds including thiodipropionic acid and its esters and salts, thio-bis (ethylene glycol beta-aminocrotonate), cysteine, cystine and methionine; and nitrogen-containing compounds including primary, secondary and tertiary amines and their derivatives.

The compositions may be activated or “triggered” (i.e. brought to their oxygen scavenging form) as required by treatment with, for example, light of a certain intensity or wavelength (e.g. TV light) or, alternatively, by the application of heath, γ-irradiation, corona discharge or an electron beam. Such treatments reduce the epoxy anthraquinonesulfonamide compound(s)/radical(s) present in the composition by conversion to an excited state such as a triplet form, which is then reduced by abstracting an electron or hydrogen atom from another molecule (e.g. a hydrogen donor compound) in the composition or by redistributing an electron or hydrogen atom within the epoxy anthraquinonesulfonamide compound(s)/radical(s) structure(s). The reduced epoxy anthraquinonesulfonamide compound(s)/radical(s) is reactive towards molecular oxygen to produce activated species such as hydrogen peroxide, hydroperoxy radical or a superoxide ion.

Compositions according to the fourth aspect may be in a solid, semisolid (e.g. a gel) or liquid form. They may therefore be applied as, or incorporated in, for example, bottle closure liners, inks, coatings, adhesives (e.g. polyurethanes), films, sheets or layers in containers such as trays or bottles either alone or as laminations or co-extrusions. When used in films or layers, they may be blended with typical polymers and/or copolymers used for construction of films or layers such as those approved for food contact. Such films or layers may be produced by extrusion at temperatures between 50° C. and 350° C. depending upon chemical composition and molecular weight distribution.

The compounds, reaction products and compositions can also be used to reveal leaks in packages or to indicate package damage caused by handling or tampering. That is, the compounds, reaction products and compositions of the invention may undergo an indicative change in colour or change in UV-visible, infrared or near-infrared absorption spectrum, as the capacity for scavenging oxygen becomes exhausted.

In a fifth aspect, the present invention provides a method of scavenging oxygen (particularly ground state oxygen) in an enclosed atmosphere or liquid comprising the steps of:

(i) treating a composition according to the fourth aspect with predetermined conditions so as to reduce the epoxy anthraquinonesulfonamide compound(s)/radical(s) to a reduced form oxidizable by oxygen; and

(ii) exposing the atmosphere or liquid to said composition, such that at least a portion of the oxygen in the enclosed atmosphere or liquid is removed through oxidation of the reduced form of the epoxy anthraquinonesulfonamide compound(s)/radical(s).

The steps (i) and (ii) may be carried out in either order.

The terms “comprise”, “comprises” and “comprising” as used throughout the specification are intended to refer to the inclusion of a stated step, component or feature or group of steps, components or features with or without the inclusion of a further step, component or feature or group of steps, components or features.

In the specification, unless stated otherwise, where a document, act or item of knowledge is referred to or discussed, that reference or discussion is not an admission that the document, act or item of knowledge, or any combination thereof, at the priority date, was part of the common general knowledge in the art.

The invention will now be described with reference to the following, non-limiting examples.

### EXAMPLE 1

### 2-(N,N-diglycidyl)-anthraguinonesulfonamide M)

A. Preparation of 2-anthraquinonesulfonyl chloride

A suspension of 2-anthraquinonesulfonic acid sodium salt monohydrate (500 g, 1.52 mol) in thionyl chloride (1 L, 13.71 mol) was stirred at room temperature for 30 minutes. The suspension was taken to reflux and DMF (25 ml, 0.32 mol) added dropwise. After stirring at reflux for 3 hours the excess thionyl chloride was removed in vacuo and the residue triturated by stirring rapidly in water (5 L). The resulting precipitate was collected, washed liberally with warm water and air dried prior to drying in vacuo at 40° C. to give 2-anthraquinonesulfonyl chloride (420 g, 90%).

B. Preparation of 2-anthraquinonesulfonaride

To a refluxing ammonia solution (specific gravity 0.91; 2.5 L), 2-anthraquinonesulfonyl chloride (180 g, 0.59 mol) was added. The resulting suspension was maintained at reflux for 20 minutes. Whilst still hot the crude precipitate was collected, washed with liberal volumes of chilled methanol, water and a further portion of chilled methanol and air dried prior to drying in vacuo at 40° C. to give 2-anthraquinonesulfonamide (148 g, 88%). The product can be further purified prior to subsequent synthetic steps via recrystallisation from methoxyethanol; m.p. 258.5-261.3° C.

C. Preparation of 2-(N,N-diglycidyl)-anthraquinonesulfonamide

To a solution of 2-anthraquinonesulfonamide (77.5 g, 0.31 mol) in NMP (225 ml) at 55° C. was added dropwise 50% aqueous NaOH (98.4 g, 2.46 mol) over 15 mins. The reaction mixture was stirred for 10 minutes before epichlorohydrin (485 ml, 6.2 mol) was added. After stirring for a further 5 hours at 55° C., the reaction mixture was diluted by the addition of methanol (675 ml). The diluted mixture was allowed to cool to room temperature overnight precipitating a yellow solid. The solid was collected by filtration, washed successively with chilled methanol, water and a further portion of chilled methanol, and dried in vacuo at 40° C. to yield (76;5 g, 62%) of 2-(N, N-diglycidyl)-anthraquinonesulfonamide. The product was recrystallised from chloroform/methanol; m.p. 164.5-164.9° C. ¹H NMR (200 MHz, CDCl₃) 2.6 (2H, m), 2.85 (2H, m), 3.1-4.0 (6H, m), 7.8-8.7 (7H, m) ppm. Selected ¹³C NMR (200 MHz, CDCl₃) 45.27, 45.32, 50.17 (CH), 50.53 (CH), 51.09, 51.62 ppm. The structure of this compound is shown as formula VI in FIG. 1.

### EXAMPLE 2

### 2-(N-butyl-N-glycidyl)-anthraquinonesulfonamide (VIII)

A. Preparation of 2-(N-butyl)-anthraquinonesulfonamide

To a solution of n-butylamine (4.69 ml, 29 mmol) in 2-methoxyethanol (9 ml) at 0° C. was added 2-anthraquinonesulfonyl chloride (3 g, 9.8 mmol). After stirring for 2 hours at 0° C., the reaction was allowed to warm to ambient temperature. The resulting precipitate was collected, washed successively with chilled methanol, water and a further portion of chilled methanol, and dried in vacuo at 50° C. to yield, (2.39 g, 71%) of 2-(N-butyl)-anthraquinonesulfonamide as a yellow coloured solid; m.p. 162.9-163.4° C.

¹H NMR (200 MHz, CDCl₃) 0.85 (3H, t), 1.4 (4H, m), 3.05 (2H, q), 4.90 (1H, t), 7.7-8.75 (7H, m) ppm.

B. Preparation of 2-(N-butyl-N-glycidyl)-anthraquinonesulfonamide

To a solution of 2-(N-butyl)-anthraquinonesulfonamide (log, 30 mmol) and epichlorohydrin (23.5 ml, 0.3 mol) in NMP(30 ml) at 80° C. was added tetrabutylammonium bromide (935 mg, 2.9 mmol). After stirring at 80° C. for 1.5 hours, the reaction mixture was cooled to 50° C. and then 50% aqueous NaOH (3.5 g, 88 mmol) was added dropwise. The reaction mixture was maintained at 50° C. overnight. The reaction mixture was cooled to room temperature and diluted with methanol (−220 ml). The resulting yellow precipitate was collected and washed several times with water and finally methanol. The isolated product was dried in vacuo to yield (8.3 g, 69%) of 2(N-butyl-N-glycidyl)-anthraquinonesulfonamide; m.p 121.8-122.9° C. ¹H NMR (200 MHz, CDCl₃) 0.95 (3H, t), 1.35 (2H, m), 1.6 (2H, m), 2.6 (1H, dd), 2.8 (1H, dd), 2.95-3.5 (4H, m), 3.8 (1H, dd), 7.8-8.7 (7H, m) ppm. Selected. ¹³C NMR (200 MHz, CDCl₃) 13.64 (CH₃), 19.77, 30.46, 45.18, 49.1, 50.67 (CH), 50.74 ppm. The structure of 2-(N-butyl-N-glycidyl)-anthraquinonesulfonamide is shown as formula VIII in FIG. 1.

### EXAMPLE 3

### 2,6-(N,N′-dibutyl-N,N′-diglycidyl)-anthraquinonesulfonamide (VII)

A. Preparation of 2,6-anthraquinonedisulfonyl Chloride

A suspension of 2,6-anthraquinonedisulfonic acid disodium salt (177 g, 0.43 mol) in thionyl chloride (350 ml, 4.8 mol) was stirred at room temperature initially for 30 minutes. The suspension was taken to reflux and DMF (9 ml) added dropwise. The suspension/solution was held at reflux for approximately 2.5 hours and terminated by the careful addition of water (6 L). The resultant precipitate was collected, washed further with hot water (2 L) and ethanol (1 L), and air dried prior to drying in vacuo at 50° C. to give 2,6-anthraquinonedisulfonyl chloride (157 g, 91%).

B. Preparation of 2,6-(N,N′-dibutyl)-anthraquinonesulfonamide

To a solution of n-butylamine (500 ml, 5 mol) in 2-methoxyethanol (1.5 L) was added 2,6-anthraquinonedisulfonyl chloride (100 g, 0.25 mol). After stirring for 2 hours at room temperature, the reaction mixture was poured into water (6 L). The resulting precipitate was collected by filtration, washed with water and then steeped in methanol (1 L) overnight. The product was collected, washed with ethanol (1 L) and dried in vacuo at 50° C. to yield (109.8 g, 92%) of 2,6-(N,N′-dibutyl)-anthraquinonesulfonamide as a beige coloured solid; m.p. 291.7-292.8° C. ¹H NMR (200 MHz, DMSO-d⁶) 0.85 (6H, d), 1.35 (8H, m), 2.85 (4H, m), 8.1 (2H, t), 8.3 (2H, d), 8.45 (2H, d), 8.55 (2H, s) ppm.

C. Preparation of 2,6-(N,N-dibutyl-N,N′-diglycidyl)-anthraquinonesulfonamide

To a solution of 2,6-(N,N′-dibutyl)-anthraquinonesulfonamide (1 g, 2.09 mmol), epichlorohydrin (3.3 ml, 41.8 mmol) in NMP (3 ml) at 80° C. was added tetrabutylammonium bromide (135 mg, 0.42 mmol). After stirring at 80° C. for 1.5 hours, the reaction mixture was cooled to 50° C. and then 50% aqueous NaOH (0.5 g, 12.5 mmol) was added in one portion. The reaction mixture was maintained at 50° C. overnight. The reaction mixture was cooled to room temperature prior to diluting with methanol (16 ml). The resulting yellow precipitate was collected and washed several times with water and finally methanol. The isolated product was dried in vacuo at 50° C. to yield (398 mg, 33%) of 2,6-(N,N′-dibutyl-N,N′-diglycidyl)-anthraquinonesulfonamide; m.p 239.7-240.1° C.: ¹H NMR (200 MHz, DMSO-d⁶) 0.90 (6H, t), 1.30 (4H, m), 1.6 (4H, m), 2.55 (2H, dd), 2.75 (2H, dd), 2.9-3.4 (8H, m), 3.65 (2H, dd), 8.4-8.6 (6H, m) ppm. The structure of this compound is shown as formula VII in FIG. 1.

### EXAMPLE 4

### Reaction Product from 2-(N,N-diglycidyl-anthraquinonesulfonamide (VI) and Ethanolamine

To a 150 ml resin kettle equipped with overhead stirring, nitrogen inlet and a thermocouple were added ethanolamine (0.91 g, 14.96 mmol) and 2-(N,N-diglycidyl)-anthraquinonesulfonamide (5.98 g, 14.96 mmol). To this mixture was added Dowanol DPM (10 ml) and NMP (6 ml). The contents of the flask were heated gradually to about 150° C. over 1 hour. The resulting dark amber coloured reaction mixture was maintained at 150° C. for 1.5 hours. Diethanolamine (0.32 g) in DMF (5 ml) was then added and the reaction stirred for a further 30 mins. After dilution with DMF (6.5 ml), the product was isolated by precipitation into a vigorously stirred 8:3:1 ice/water/methanol mixture yielding a fine brown precipitate. After filtration and washing with water, the product was steeped for 64 hours in 4:1 water/methanol. Finally, the product was collected, washed with water and air-dried for 6 hours. The product was then dried in vacuo at 50° C. for 18 hours to yield (5.52 g) of a yellow powder. The degree of anthraquinone concentration was determined by WV-VIS spectroscopy in DMSO at 326 nm (ε=5712 lmol⁻¹cm⁻¹) to be 42.9% w/w.

### EXAMPLE 5

### Reaction Product from 2-(N,N-diglycidyl)-anthraquinonesulfonamide (VI), Ethanolamine and Bisphenol-A Diglycidyl Ether

To a 150 ml resin kettle equipped with overhead stirring, nitrogen inlet and a thermocouple were added bisphenol-A diglycidyl ether (19.83 g, 57.63 mmol), ethanolamine (3.74 g, 61.15 mmol) and 2-(N,N-diglycidyl)-anthraquinonesulfonamide (1.44 g, 3.61 mmol). To this mixture was added Dowanol DPM (27 ml) as solvent. The contents of the flask were heated gradually to about 150° C. over 1 hour. The resulting viscous amber coloured reaction mixture was maintained at −150° C. for 4 hours. Diethanolamine (0.36 g) in DMF (20 ml) was then added and the reaction stirred for a further 30 mins. After dilution with DMF (17 ml), the product was isolated by precipitation into a vigorously stirred 8:3:1 ice/water/methanol mixture and steeped for 16 hours. After filtration and washing with water, the product was twice washed with 8:3:1 ice/water/methanol in the high-speed blender, collected and steeped for 21 hours in 4:1 water/methanol. The product (21 g), after filtration, was then reprecipitated from DNT (125 ml) into a vigorously stirred 8:3:1 ice/water/methanol mixture. The product was collected, washed with water and further washed with 8:3:1 ice/water/methanol in the high-speed blender. The yellow solid obtained was collected washed with thoroughly with water and steeped in 3:1 water/methanol for 24 hours. Finally, the product was collected, washed with water and air-dried for 6 hours. The product was then dried in vacuo at 55° C. for 17 hours to yield (20.4 g) of a light yellow powder. The degree of anthraquinone concentration was determined by UV-VIS spectroscopy in DMSO at 324 nm (E=5712 lmol⁻¹cm⁻¹) to be 2.83% w/w.

### EXAMPLE 6

### Reaction Product from 2-(N-butyl-N-glycidyl)-anthraquinonesulfonamide (VIII) and Poly(ethylene-co-acrylic Acid)

To a solution of a commercially available poly(ethylene-co-acrylic acid) (Primacor 5980i, Dow Chemical Co., USA) (2 g, 4.65 mmol of acrylic acid units) in toluene (45 ml) and 2-propanol (5 ml) at reflux was added 2-(N-butyl-N-glycidyl)-anthraquinonesulfonamide (1 g, 2.5 mmol) followed by tetrabutylammonium bromide (160 mg, 0.5 mmol). After stirring at reflux for 20 hours the product was precipitated from methanol (200 ml). The resulting fine yellow precipitate was collected and washed successively with methanol, water, acetone and finally methanol. The product was dried in vacuo at 55° C. for 17 hours. The degree of anthraquinone concentration was determined by WV-VIS spectroscopy in chloroform at 329 nm (E=5712 lmol⁻¹cm⁻¹) to be 13.7% w/w.

### EXAMPLE 7

### Oxygen Scavenging by a Composition Comprising a Product from Reaction of 2-(N,N-diglycidyl)-anthraquinonesulfonamide (VI) and Ethanolamine, and Poly(hydroxyaminoether)

A composition was prepared by blending a polymeric product of the reaction of 2-(N,N-diglycidyl)-anthraquinonesulfonamide and ethanolarmine (prepared according to the method described in Example 4) into a commercially available poly(hydroxyaminoether) (BLOX™, Dow Chemical Co., USA) at a level of 5% w/w of the polymeric reaction product. The composition was then compression molded to form a film having a thickness of about 60 μm. This film was placed between two layers of polypropylene film and vacuum sealed to form a flat package containing essentially no headspace. The package was placed on a conveyor belt moving at 10 n/min and then exposed to light from a commercial UV-curing lamp (model F-300 fitted with a ‘D’ bulb (Fusion Systems Corp., Maryland, USA)). After exposure to the lamp, the package was opened and the film was then quickly transferred into a foil multilayer bag, and this bag was then vacuum sealed to form a flat package containing essentially no headspace. This foil-lined pouch allows essentially no ingress of oxygen from the atmosphere into the inside of the pouch. Air was then injected into the foil-lined pouch and the pouch stored at 60° C. The oxygen content inside the pouch was measured by gas chromatography. The reduction in oxygen content of two pouches prepared in the manner described, are shown in Table 1.

TABLE 1

Oxygen content
inside pouch (%)

| Time (days) | Pouch 1 | Pouch 2 |
|---|---|---|
| 0 | 20.9 | 20.9 |
| 1 | 15.8 | 15.6 |
| 2 | 14.8 | 14.1 |
| 7 | 10.4 | 8.9 |
| 14 | 6.1 | 5.2 |

### EXAMPLE 8

### Photoreduction, Re-Oxidation and Oxygen Scavenging by a Composition Comprising a Product from Reaction of 2-(N,N-diglycidyl)-anthraquinonesulfonamide (VI), Ethanolamine and Bisphenol-A Diglycidyl Ether

The polymeric product of the reaction of 2-(N,N-diglycidyl)-anthraquinesulfonamide and ethanolamine and bisphenol-A diglycidyl ether (prepared according to the method described in Example 5) was compression molded to form a film having a thickness of about 50 μm. This film was placed between two layers of polypropylene film and vacuum sealed to form a flat package containing essentially no headspace. The package was placed on a conveyor belt moving at 10 m/min and then exposed to light from a commercial UV-curing lamp (model F-300 fitted with a ‘D’ bulb (Fusion Systems Corp. Maryland, USA)). After exposure to the lamp, the package was opened and the film was then quickly transferred into a foil multilayer bag, and this bag was then vacuum sealed to form a flat package containing essentially no headspace. This foil-lined pouch allows essentially no ingress of oxygen from the atmosphere into the inside of the pouch. Air was then injected into the foil-lined pouch and the pouch stored at 60° C. The oxygen content inside the pouch was measured by gas chromatography. The reduction in oxygen content of two pouches prepared in the manner described, are shown in Table 2.

TABLE 2

Oxygen content
inside pouch (%)

| Time (days) | Pouch 1 | Pouch 2 |
|---|---|---|
| 0 | 20.9 | 20.9 |
| 1 | 15.3 | 15.6 |
| 2 | 13.2 | 13.2 |
| 7 | 5.4 | 6.0 |
| 14 | 2.0 | 1.9 |

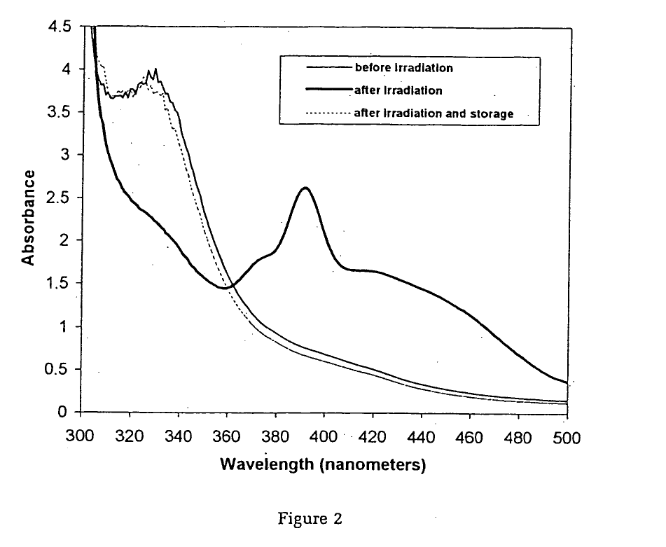

The absorption spectrum of a film prepared from this composition before to the lamp, immediately after exposure to the lamp, and after exposure to the lamp followed by storage in air at 60° C. for 7 days are shown in FIG. 2. These spectra clearly illustrate that photoreduction occurs on exposure to the lamp, followed by reoxidation upon exposure to air.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An epoxy anthraquinonesulfonamide compound of the following formula: 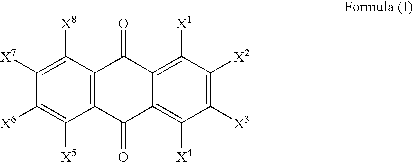
wherein X¹, X², X³, X⁴, X⁵, X⁶, X⁷ and X⁸ are each independently selected from H, OH, NH₂, C₁-C₄₀ alkyl, C₁-C₄₀ alkoxy, C₁-C₄₀ alkanoyl, C₁-C₄₀ alkanol, C₁-C₄₀ alkylether, C₁-C₄₀ alkylthio, C₁-C₄₀ alkylamino, C₁-C₄₀alkanolether, C₁-C₄₀ alkylaminoether, C₁-C₄₀ alkylsulfonyl, C₁-C₄₀ alkyl sulfonamido, epoxy sulfonamido substituents, and sulfonate substituents, with the proviso that at least one of X¹, X², X³, X⁴, X⁵, X⁷ and X⁸ is an epoxy sulfonamido substituent,
and salts thereof.

2. A compound according to claim 1, wherein X¹, X², X³, X⁴, X⁵, X⁶, X⁷ and X⁸ are each independently selected from H, OH, NH₂, C₁-C₁₀ alkyl, C₁-C₂₀ alkoxy, C₁-C₂₀ alkanoyl, C₁-C₂₀ alkanol, C₁-C₂₀ alkylether, C₁-C₂₀ alkylthio, C₁-C₂₀ alkylamino, C₁-C₂₀ alkanolether, C₁-C₂₀ alkylaminoether, C₁-C₂₀ alkylsulfonyl, C₁-C₂₀ alkyl sulfonamido, epoxy sulfonamido substituents, and sulfonate substituents, with the proviso that at least one of X¹, X², X³, X⁴, X⁵, X⁶, X⁷ and X⁸ is an epoxy sulfonamido substituent,

3. A compound according to claim 1 or 2, wherein the epoxy sulfonamido substituent(s) is/are selected from those having the following formula: 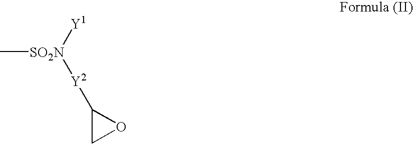
wherein Y¹ is selected from H, C₁-C₄₀ alkyl, C₁-C₄₀ alkanol, C₁-C₄₀ alkanolether, C₁-C₄₀ alkylamine, C₁-C₄₀ alkylaminoether, C₁-C₄₀alkylether, C₁-C₄₀ aryl, C₁-C₄₀ arylalkyl and 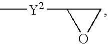
and Y² is selected from C₁-C₂₀ alkyl.

4. A compound according to claim 3, wherein Y¹ is selected from H, C₁-C₂₀ alkyl, C₁-C₂₀ alkanol, C₁-C₂₀ alkanolether, C₁-C₂₀ alkylamine, C₁-C₂₀ alkylaminoether, C₁-C₂₀ alkylether, C₁-C₂₀ aryl, C₁-C₂₀ arylalkyl and

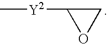

5. A compound according to claim 3 or 4, wherein Y² is selected from C₁-C₈ alkylene.

6. A compound according to any one of claims 3 to 5, wherein Y² is methylene.

7. A compound according to any one of claims 1 to 5, wherein only one of X¹, X², X³ and X⁴, and/or one of X⁵, X⁸, X⁷ and X⁸ is/are an epoxy sulfonamido substituent(s).

8. A compound selected from the group consisting of 2-(N,N-diglycidyl)-anthraquinonesulfonamide, 2-(N-butyl-N-glycidyl)-anthraquinonesulfonamide and 2,6-(N,N′-dibutyl-N,N′-diglycidyl)-anthraquinonesulfonamide.

9. A reaction product of a compound according to any one of claims 1 to 8 and a dinucleophilic compound.

10. A product according to claim 9, wherein the dinucleophilic compound is selected from primary amines, bis(secondary) diamines, dihydric phenols, dicarboxylic acids, anhydrides, diols, dithiols and disulfonamides.

11. A product according to claim 9 or 10, wherein the product is a compound of the following formula:
B¹-[-(Z¹-Aⱼ^{l})ₖ-(Z²-Aⱼ²)ₗ- . . . -(Zⁿ-Aⱼⁿ)ₘ-]-B² Formula [III]
wherein Z¹, Z², . . . and Zⁿ are each independently selected from the group consisting of radicals derived from di-epoxy compounds, and where at least Z¹ is selected from the group consisting of radicals derived from an epoxy anthraquinonesulfonamide compound according to any one of claims 1 to 8,
A¹, A², . . . and Aⁿ are each independently selected from the group consisting of radicals derived from dinucleophilic compounds selected from primary amines, bis(secondary) diamines, dihydric phenols, dicarboxylic acids, anhydrides, diols, dithiols and disulfonamides,
B¹ is selected from the group consisting of an epoxy group, or remnants of an epoxy group after reaction with Aⁿ, or suitable capping groups,
B² is selected from the group consisting of H, or an epoxy group, or radicals derived from di-epoxy compounds or their remnants after reaction with Aⁿ, or suitable capping groups,
k is 1, l is 0 or 2, m is 0 or any integer in the range of 3 and 1000, and n is equal to m, wherein when m is other than 0, l must be 2, and
j is 0 or 1 when l and m are 0, and j is 1 when l and m are other than 0.

12. A reaction product of a compound according to any one of claims 1 to 8 and a compound containing one or more nucleophilic groups.

13. A product according to claim 12, wherein the nucleophilic compound contains one or more amine, acid, anhydride, phenol, alcohol, thiol or sulfonamide groups.

14. An oxygen scavenging composition comprising a compound according to any one of claims 1 to 8 or a product according to any one of claims 9 to 13.

15. A composition according to claim 14, further comprising a hydrogen donor compound.

16. A composition according to claim 15, wherein the hydrogen donor compound is a compound containing a hydrogen bonded to a nitrogen, sulfur, phosphorus or oxygen.

17. A composition according to claim 16, wherein the hydrogen donor compound is selected from salts of organic compounds.

18. A composition according to any one of claims 14 to 17, further comprising an activated oxygen scavenging agent.

19. A composition according to claim 18, wherein the activated oxygen scavenging agent is selected from organic antioxidants, organic phosphites, organic phosphines, organic phosphates, hydroquinones and substituted hydroquinones; inorganic compounds, nitrites of metals, sulphur-containing compounds, and nitrogen-containing compounds.

20. A method of scavenging oxygen in an enclosed atmosphere or liquid comprising the steps of:
(i) treating a composition according to any one of claims 14 to 19 with predetermined conditions so as to reduce the epoxy anthraquinonesulfonamide compound(s)/radical(s) to a reduced form oxidizable by oxygen; and
(ii) exposing the atmosphere or liquid to said composition, such that at least a portion of the oxygen in the enclosed atmosphere or liquid is removed through oxidation of the reduced form of the epoxy anthraquinonesulfonamide compound(s)/radical(s), wherein steps (i) and (ii) are carried out in either order.
